(19) United States
(12) Patent Application Publication
(10) Pub. No.: US 2019/0160105 A1
(43) Pub. Date: May 30, 2019

(54) METHODS FOR SELECTING IMPROVED STEM CELL FOR TREATING INTRAVENTRICULAR HEMORRHAGE OF PREMATURE INFANTS

(71) Applicant: SAMSUNG LIFE PUBLIC WELFARE FOUNDATION, Seoul (KR)

(72) Inventors: Yun Sil CHANG, Seoul (KR); Won Soon PARK, Seoul (KR); So Yoon AHN, Seoul (KR); Dong Kyung SUNG, Seoul (KR)

(73) Assignee: SAMSUNG LIFE PUBLIC WELFARE FOUNDATION

(21) Appl. No.: 16/205,372

(22) Filed: Nov. 30, 2018

(30) Foreign Application Priority Data
Nov. 30, 2017 (KR) 10-2017-0163731

(51) Int. Cl. A61K 35/28; A61P 9/14; G01N 33/50

(52) U.S. Cl. CPC A61K 35/28; G01N 2333/475; G01N 33/5073; A61P 9/14

(57) ABSTRACT

The present disclosure relates to a method for selecting a high efficacy stem cell for treating intraventricular hemorrhage in premature infants, and more particularly, to a method for selecting a high efficacy stem cell for treating intraventricular hemorrhage, including a step of measuring an expression level of a vascular endothelial growth factor (VEGF) and a high efficacy stem cell selected by the method.

### CROSS-REFERENCE TO RELATED APPLICATION

This application claims priority to and the benefit of Korean Patent Application No. 2017-0163731, filed on Nov. 30, 2017, the disclosure of which is incorporated herein by reference in its entirety.

### FIELD

The present disclosure relates to a method for selecting a high efficacy stem cell for treating intraventricular hemorrhage in premature infants, and more particularly, to a method for selecting a high efficacy stem cell for treating intraventricular hemorrhage, including a step of measuring an expression level of a vascular endothelial growth factor (VEGF), a high efficacy stem cell selected by the method, and a use thereof.

### BACKGROUND

Intraventricular hemorrhage (IVH) usually occurs due to arteriovenous malformation, rupture of cerebral aneurysm, thalamic hemorrhage, or brain basal ganglia hemorrhage, and is a disease in which deep disorder of consciousness, quadriplegia, abnormalities of breathing, febricity, pinhole pupil, diaphoresis, other autonomic nervous system symptoms, and the like occur. Intraventricular hemorrhages can be observed by computed tomography (CT) and thus may be appropriately treated in the early stage but are a major disease which still causes deaths and neurological disorders in newborns, particularly, premature infants, and there are few effective treatments in practice. Accordingly, the development of a new treatment for intraventricular hemorrhage is an extremely urgent and important issue in improving such a serious prognosis.

In this regard, recently, the present inventors have confirmed that intraventricular transplantation of human umbilical cord blood-derived mesenchymal stem cells in newly born white rats remarkably reduces posthemorrhagic hydrocephalus and brain damage caused by severe intraventricular hemorrhage. Further, it has been reported that transplantation of mesenchymal stem cells also has a remarkable treatment effect on various diseases such as bronchopulmonary dysplasia, acute respiratory distress syndrome, and neonatal stroke through anti-inflammation and apoptosis-suppressive effects caused by paracrine rather than a mechanism caused by regeneration.

Meanwhile, it has been reported that various growth factors, for example, a brain-derived neurotrophic factor (BDNF), a nerve growth factor (NGF), a vascular endothelial growth factor (VEGF), insulin-like growth factor(IGF), interleukins, and the like improve brain damage recovery ability after hypoxia and/or ischemia. However, during severe intraventricular hemorrhage, effects of a specific paracrine factor, particularly, VEGF exhibiting neuroprotective effects by transplantation of mesenchymal stem cells, and the mechanism thereof, have not yet been elucidated.

### SUMMARY

As a result of studies to elucidate effects of a vascular endothelial growth factor (VEGF) and the action mechanism thereof in the treatment of cerebrovascular diseases using mesenchymal stem cells, the present inventors confirmed that the VEGF is an important factor in treating intraventricular hemorrhage using mesenchymal stem cells by confirming that when the expression of the VEGF is inhibited, treatment effects of intraventricular hemorrhage by transplantation of mesenchymal stem cells are not exhibited, and symptoms, such as increases in reactive gliosis and apoptosis in tissues around the cerebral ventricle, a decrease in myelination, and an increase in encephalitis, are not alleviated and experimentally elucidated a correlation between the expression level of VEGF and an ability to protect nerve cells in the mesenchymal stem cells, thereby completing the present disclosure.

Thus, the present disclosure provides a method for selecting a high efficacy stem cell for treating a cerebrovascular disease, the method including a step of measuring a level of a VEGF and a high efficacy stem cell selected by the method.

Further, the present disclosure provides a pharmaceutical composition for treating a cerebrovascular disease which includes the high efficacy stem cell.

However, a technical problem to be solved by the present disclosure is not limited to the aforementioned problem, and other problems that are not mentioned may be clearly understood by a person skilled in the art from the following description.

The present disclosure provides a method for selecting a high efficacy stem cell, the method including a step of measuring an expression level of a VEGF.

As an embodiment of the present disclosure, the present disclosure may include the steps of: (a) culturing stem cells; (b) measuring a concentration of a VEGF in a culture solution of the stem cells in Step (a); and (c) evaluating an ability to protect nerve cells based on the measured concentration.

As another embodiment of the present disclosure, the evaluation of the ability to protect nerve cells may be determined to be high efficacy when the measured concentration of the VEGF is 50 pg/ml or more.

As still another embodiment of the present disclosure, the evaluation of the ability to protect nerve cells may be determined to be high efficacy when the measured concentration of the VEGF is 100 pg/ml or more.

Further, the present disclosure provides a high efficacy stem cell for treating a cerebrovascular disease selected by the selection method.

In addition, the present disclosure provides a pharmaceutical composition for treating a cerebrovascular disease which includes the high efficacy stem cell.

As an embodiment of the present disclosure, the cerebrovascular disease may be intraventricular hemorrhage (IVH) in a newly born baby.

As another embodiment of the present disclosure, the high efficacy may be an ability to protect nerve cells.

As still another embodiment of the present disclosure, the stem cell may be a stem cell selected from the group consisting of a mesenchymal stem cell, a human tissue-derived mesenchymal stromal cell, a human tissue-derived mesenchymal stem cell, a multipotent stem cell, and an amniotic epithelial cell.

As yet another embodiment of the present disclosure, the mesenchymal stem cell may be derived from umbilical cord, umbilical cord blood, bone marrow, fat, muscles, nerves, skin, amnion, or placenta.

Furthermore, the present disclosure provides a method for treating a cerebrovascular disease, the method including a step of administering the pharmaceutical composition to an individual.

Further, the present disclosure provides a use of the pharmaceutical composition for treating a cerebrovascular disease.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a verification of the function of a vascular endothelial growth factor (VEGF) small interfering Ribonucleic Acid (siRNA), and a result of measuring, after umbilical cord-derived mesenchymal stem cells are transfected with the VEGF siRNA, the level of the VEGF in a culture solution of the umbilical cord-derived mesenchymal stem cells over time;

FIG. 2 is a result of comparing and analyzing the degree of ventricular enlargement by brain magnetic resonance imaging (MRI) among a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC);

FIG. 3A is a result of evaluating a sensorimotor function, and a result of evaluating a negative geotaxis among a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC);

FIG. 3B is a result of evaluating a sensorimotor function, and a result of evaluating a rotarod among a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC);

FIG. 4 is a result obtained by respectively performing reactive gliosis (GFAP), apoptosis (TUNEL), and myelination (MBP) analyses on a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC);

FIG. 5A is a result of measuring the number of ED-1-positive cells in order to analyze the induction of inflammation of tissues around the cerebral ventricle among a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC);

FIG. 5B is a result of analyzing the expression levels of inflammatory cytokines (IL-1a, IL-1b, IL-6, and TNF-α) among a control in which intraventricular hemorrhage is caused (IVH+Saline), a general mesenchymal stem cell transplantation group (IVH+MSC), a transplantation group of mesenchymal stem cells transfected with a scrambled siRNA (IVH+Scrambled siRNA MSC), and a group into which mesenchymal stem cells, in which the expression of the VEGF is inhibited, are transplanted (IVH+VEGF KD MSC); and

FIG. 6 is a result of analyzing a correlation between the VEGF level and the viability of nerve cells in order to predict/select a high efficacy stem cell.

### DETAILED DESCRIPTION

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the invention.

The present inventors confirmed that when the expression of a vascular endothelial growth factor (VEGF) was inhibited, the treatment effects of intraventricular hemorrhage by mesenchymal stem cell transplantation were not exhibited, and increases in reactive gliosis and apoptosis in tissues around the cerebral ventricle, a decrease in myelination, an increase in encephalitis, and the like are not alleviated, and experimentally confirmed that the VEGF is an important factor in treating intraventricular hemorrhage by mesenchymal stem cells, thereby completing the present disclosure.

Thus, the present disclosure provides a method for selecting a high efficacy stem cell, including a step of measuring an expression level of a VEGF, and a high efficacy stem cell selected by the method.

In the present disclosure, the method is characterized by including the steps of: (a) culturing stem cells;

(b) measuring a concentration of a VEGF in a culture solution of the stem cells in Step (a); and

(c) evaluating an ability to protect nerve cells based on the measured concentration.

The present inventors classified newly born white rats into 5 groups, that is, a normal control (NC), a control in which intraventricular hemorrhage was caused (IVH+Saline), a general mesenchymal stem cell transplantation group (IVH+MSC), a transplantation group of mesenchymal stem cells transfected with a scrambled small interfering Ribonucleic Acid (siRNA) (IVH+Scrambled siRNA MSC), and a group into which mesenchymal stem cells, in which the expression of the VEGF was inhibited, were transplanted (IVH+VEGF KD MSC) in the Examples, and experimentally proved that a high efficacy stem cell could be selected by measuring the levels of the VEGF in the groups through various experiments.

In an Example of the present disclosure, as a result of measuring the degree of ventricular enlargement by performing brain magnetic resonance imaging (MRI) on the 5 groups to calculate the ratio of volume of the entire cerebral ventricle/volume of the entire brain, it was confirmed that when mesenchymal stem cells, in which the expression of the VEGF was inhibited, were transplanted, the degree of ventricular enlargement was remarkably increased unlike the general mesenchymal stem cell group and the transplantation group of mesenchymal stem cells transfected with the scrambled siRNA (see Example 2).

In another Example of the present disclosure, as a result of performing a negative geotaxis evaluation and a rotarod evaluation on each of the 5 groups as an evaluation of sensorimotor behaviors, it was also confirmed that when mesenchymal stem cells, in which the expression of the VEGF was inhibited, were transplanted, the damaged motor ability was not improved unlike the general mesenchymal stem cell group and the transplantation group of mesenchymal stem cells transfected with the scrambled siRNA (see Example 3).

In still another Example of the present disclosure, as a result of performing a reactive gliosis analysis, an apoptotic analysis, and a myelination analysis on the 5 groups through immunohistochemical staining and TUNNEL assay, it was confirmed that when mesenchymal stem cells, in which the expression of the VEGF was inhibited, were transplanted, increases in reactive gliosis and apoptosis and a decrease in myelination were not alleviated unlike the general mesenchymal stem cell group and the transplantation group of mesenchymal stem cells transfected with the scrambled siRNA (see Examples 4-1 to 4-3).

In yet another Example of the present disclosure, as a result of measuring the levels of inflammatory cytokines in the 5 groups by using a suspension of ED-1-positive cells of brain coronal sections and tissues around the cerebral ventricle in order to analyze whether the inflammations of the tissues around the cerebral ventricle were alleviated, decreases in levels of ED-1-positive cells and inflammatory cytokines were also not exhibited when mesenchymal stem cells, in which the expression of the VEGF was inhibited, were transplanted (see Example 5).

In still yet another Example of the present disclosure, after mesenchymal stem cells exhibiting different expression levels of the VEGF in nerve cells having induced apoptosis were treated lot by lot by preparing an in vitro model of intraventricular hemorrhage, the viability of the nerve cells according to the level of the VEGF was analyzed. As a result, it was confirmed that the higher the expression level of the VEGF was, the higher the viability of nerve cells was shown to be.

Through the Example results, it could be seen that a VEGF is a major mediating factor in allowing mesenchymal stem cells to exhibit the nerve cell protective effects in the treatment of intraventricular hemorrhage, and accordingly, it was confirmed that the ability of stem cells to protect nerve cells could be evaluated based on the concentration of the VEGF.

In the present disclosure, more preferably, in the evaluation of the ability of stem cells to protect nerve cells, a case where the measured concentration of the VEGF is 50 pg/ml or more and even more preferably, 100 pg/ml or more, may be determined to be highly efficacious.

In the present disclosure, the cerebrovascular disease may include all the diseases which can be treated by the high efficacy mesenchymal stem cell according to the present disclosure and more preferably may be intraventricular hemorrhage (IVH) in a newly born baby but is not limited thereto.

The high efficacy means an ability to protect nerve cells and includes the suppression of apoptosis and reactive gliosis of nerve cells and an increase in myelination, and furthermore, includes all recoveries of brain function, such as a decrease in ventricular enlargement and recovery of the motor ability.

The term ‘stem cell’ as used herein refers to a cell having an ability to be differentiated into two or more different cells while having a self-replication ability as an undifferentiated cell. The stem cell of the present disclosure may be an autologous or allogeneic-derived stem cell and may be derived from any type of animal including a human and a non-human mammal and is not limited to those derived from an adult body and derived from an embryo.

The stem cell of the present disclosure may be selected from the group consisting of a mesenchymal stem cell, a human tissue-derived mesenchymal stromal cell, a human tissue-derived mesenchymal stem cell, a multipotent stem cell, and an amniotic epithelial cell, and the mesenchymal stem cell may be derived from umbilical cord, umbilical cord blood, bone marrow, fat, muscles, nerves, skin, amnion, or placenta, but preferably may be an umbilical cord blood-derived mesenchymal stem cell, but is not limited thereto.

As another aspect of the present disclosure, the present disclosure provides a pharmaceutical composition for treating a cerebrovascular disease which includes the high efficacy stem cell.

The pharmaceutical composition of the present disclosure may further contain one or more publicly-known auxiliary ingredients having an effect of treating cerebrovascular disease together with the high efficacy stem cell.

A preferred dosage of the pharmaceutical composition varies depending on the condition and body weight of an individual, the degree of a disease, the form of drug, the administration route, and the duration but may be appropriately selected by a person skilled in the art. The composition may be administered once a day and may also be administered several times a day, but the administration of the composition is not limited thereto.

The pharmaceutical composition of the present disclosure may be used either alone or in combination with surgery, radiation therapy, hormone therapy, chemotherapy, and methods using a biological response modifier in order to treat a cerebrovascular disease.

The pharmaceutical composition of the present disclosure may further include an appropriate carrier, which is typically used to prepare a pharmaceutical composition. For example, an injection may further include a preservative, a soothing agent, a solubilizing agent, a stabilizer, or the like, and a preparation for topical administration may further include a base, an excipient, a lubricant, a preservative, or the like.

The composition of the present disclosure may be formulated into a preparation in a unit dosage form suitable for systemic administration to an individual, and the present disclosure may be administered depending on the typical method. As a dosage form suitable for this purpose, an injection such as an injection ampoule, an infusion such as an infusion bag, a spraying agent such as an aerosol preparation, and the like are preferred as a parenteral administration preparation. The injection ampoule may be formulated and mixed with an injection solution immediately before use, and as the injection solution, physiological saline, glucose, Ringer's solution, and the like may be used. Further, as the infusion bag, it is possible to use an infusion bag made of polyvinyl chloride or polyethylene. The “administration” as used herein refers to provision of a predetermined composition of the present disclosure to an individual by any suitable method.

In addition, the present disclosure provides a method for treating a cerebrovascular disease, the method including a step of administering a pharmaceutical composition including the high efficacy stem cell to an individual.

The “individual” as used herein refers to a subject in need of treatment of a disease, and may refer to a mammal such as a human or a non-human primate, a mouse, a rat, a dog, a cat, a horse, and a cow.

Furthermore, the present disclosure provides a use of the pharmaceutical composition for treating a cerebrovascular disease.

Hereinafter, preferred Examples for aiding in understanding of the present disclosure will be suggested. However, the following Examples are provided only to more easily understand the present disclosure, and the contents of the present disclosure are not limited by the following Examples.

### EXAMPLES

### Example 1

### Experimental Method

1-1. Preparation of Cells and VEGF siRNA Transfection

In the present disclosure, umbilical cord blood-derived mesenchymal stem cells were supplied by Medipost Co., Ltd. and used.

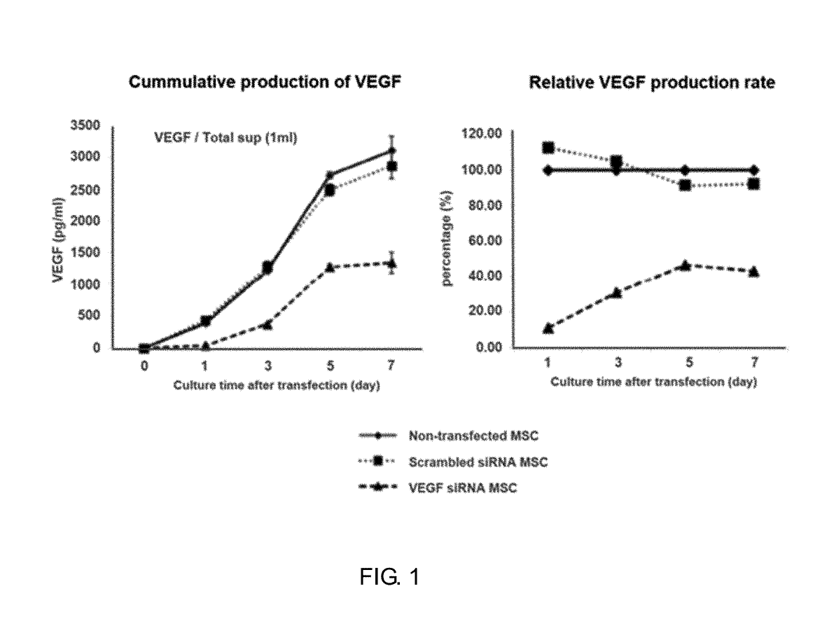

Meanwhile, a VEGF siRNA and a scrambled siRNA were each purchased from Santa Cruz Biotechnology, Inc., and the umbilical cord blood-derived mesenchymal stem cells were transfected with each of the siRNAs by using Oligofectamine (Invitrogen, Carlsbad, Calif., USA) in accordance with the manufacturer's protocol. In order to confirm whether the expression of the VEGF was inhibited by the VEGF siRNA after the transfection, the change in expression level of the VEGF was measured by collecting the culture medium of the mesenchymal stem cells over time. As a result, as illustrated in FIG. 1, it was confirmed that the expression level of the VEGF was remarkably reduced as compared to those of the non-transfected mesenchymal stem cell (MSC) and the scrambled siRNA mesenchymal stem cell (MSC) which did not target a VEGF gene.

1-2. Thrombin Treatment and Cell Culture (Thrombin Exposure In Vitro Cell Culture)

Brain nerve cells separated from undeveloped mice of E18.5 were primarily cultured, 5×10³ cells/well of the nerve cells were seeded onto a 96-well plate and then cultured at 37° C. for 24 hours by using 100 μl of a Neurobasal medium containing a B-27 supplement (GIBCO, Gaithersburg, Md., USA) per each one well. Thereafter, in order to induce neuronal damage caused by hemorrhage in vitro, the nerve cells were treated with 40 U of thrombin (Reyon pharm. Co., Ltd., Seoul, South Korea), the nerve cells treated with thrombin were cultured alone in a complete medium, or non-transfected umbilical cord blood-derived mesenchymal stem cells (1×10³), mesenchymal stem cells transfected with a scrambled siRNA or mesenchymal stem cells transfected with a VEGF siRNA were seeded in the upper chamber and co-cultured for 24 hours.

1-3. Animal Model

All experimental protocols were approved by the Institutional Animal Care and Use Committee of Samsung Biomedical Research Institute and then conducted. As experimental animals, newly born SD (Sprague-Dawley) white rats were used, and the experiment was carried out from Day 4 (P4) after birth till Day 32 (P32).

In order to induce intraventricular hemorrhage in white rats on P4, after the white rats were anesthetized with an anesthetic obtained by mixing halothane and a 2:1 mixture of nitrous oxide and oxygen, 200 μl of blood was collected from mother white rats, and 100 μl was injected into each of both of the cerebral ventricles. In order to induce intraventricular hemorrhage and confirm the degree of intraventricular hemorrhage in white rats on P5 after a day, a brain MRI was performed, and the white rats in which intraventricular hemorrhage was rarely induced or not observed by the unaided eye were excluded from the analysis. Thereafter, white rats in which intraventricular hemorrhage was caused on P6 were arbitrarily screened and classified into 5 groups as follows: a normal control (NC), a control in which intraventricular hemorrhage was caused (IVH+Saline), a general mesenchymal stem cell transplantation group (IVH+MSC), a transplantation group of mesenchymal stem cells transfected with a scrambled siRNA (IVH+Scrambled siRNA), and a transplantation group of mesenchymal stem cells transfected with a VEGF siRNA (IVH+VEGF KD MSC). While the experiment was performed, the white rats in the normal control group (NC) in which intraventricular hemorrhage was not induced all survived until P32, whereas some white rats in each group in which intraventricular hemorrhage was caused were dead and excluded from the experiment, and the experiment was performed. In order to transplant mesenchymal stem cells, 1×10⁵ of each of general mesenchymal stem cells, mesenchymal stem cells transfected with a scrambled siRNA, or mesenchymal stem cells transfected with a VEGF siRNA together with 10 μl of general saline were administered to the right cerebral ventricle of each white rat in the IVH+MSC, IVH+Scrambled siRNA, and IVH+VEGF KD MSC groups. An equal volume of saline was administered to the white rats in the IC group into which mesenchymal stem cells were not transplanted. Thereafter, a brain MRI image result for each group on P11 and P32 was obtained, and after the white rats in all the groups were euthanized on P32, the brain tissue samples were recovered.

Meanwhile, as a behavioral evaluation for evaluating sensorimotor neurons of the white rats, a negative geotaxis evaluation and a rotarod evaluation were performed.

1-4. Statistical Analyses

The sample size was measured based on the difference in volume of cerebral ventricle on P32 according to Power 0.8 and Type I error probability 0.05, which are previous study results. The experimental data was expressed as mean±standard deviation. For continuous variability, statistical comparison between the groups was carried out by using one-way ANOVA and Tukey's post hoc analysis. In order to analyze changes over time, Tukey's post hoc comparison was carried out by using the univariate general linear model for repeated measures. All the data was analyzed by using SPSS version 18.0 (IBM, Chicago, Ill., USA), and it was determined that in the case of P<0.05, there was a statistically significant difference.

### Example 2

### Brain MRI Analysis

The present inventors tried to investigate effects of a vascular endothelial growth factor (VEGF) on the nerve cell protective effects of mesenchymal stem cells against intraventricular hemorrhage based on a study result that in newly born white rats in which ischemic-hypoxic encephalopathy is caused in the related art, the functional recovery of neurons due to the introduction of a VEGF gene is improved, and the like. For this purpose, as described in Example 1-1, a method of inhibiting the expression of the VEGF in mesenchymal stem cells was used by using an siRNA specific for the VEGF, and first, in order to analyze the effects according to the inhibition of the expression of the VEGF, the white rats were caused to have intraventricular hemorrhage according to the method in Example 1-3, and then a brain MRI was performed on each group on Days 1, 7, and 28 (P5, P11, and P32).

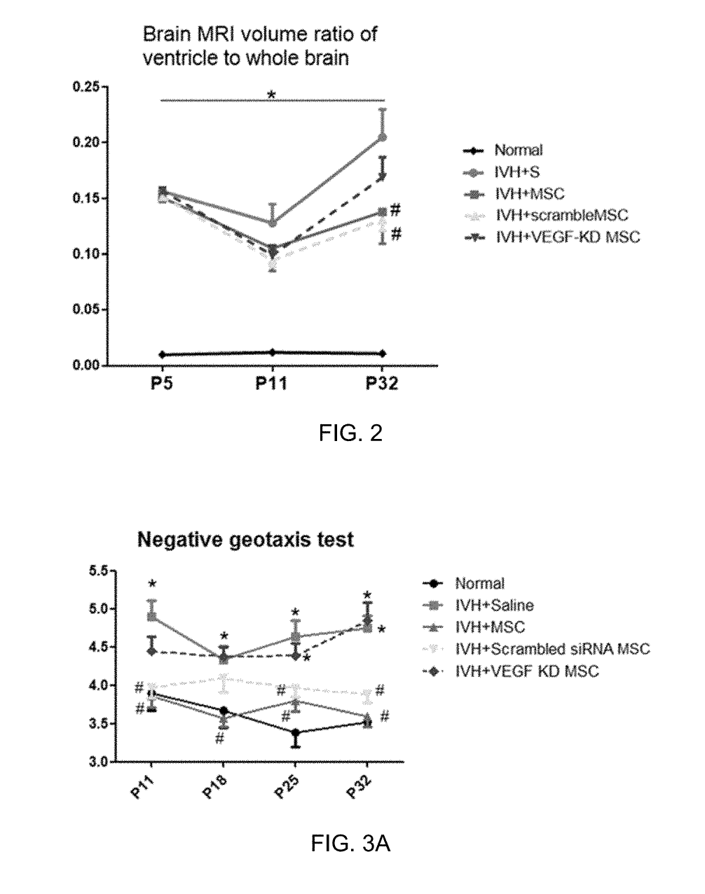

As a result of measuring the degree of ventricular enlargement by calculating each of the ratios of volume of the entire cerebral ventricle/volume of the entire brain on P5, P11, and P32, as illustrated in FIG. 2, no particular difference appeared in all the groups on P5, whereas on P11 and P32, the degree of ventricular enlargement was remarkably increased in the case of the control in which intraventricular hemorrhage was caused (IVH+Saline) and the group into which mesenchymal stem cells, in which the expression of the VEGF was inhibited, were transplanted (IVH+VEGF KD MSC). In contrast, in the case of the general mesenchymal stem cell transplantation group (IVH+MSC) or the transplantation group of mesenchymal stem cells transfected with the scrambled siRNA (IVH-scrambled siRNA MSC), it was confirmed that the degree of ventricular enlargement was significantly decreased.

### Example 3

### Analysis of Evaluation of Sensorimotor Behaviors

In addition to the result in Example 2, a negative geotaxis evaluation and a rotarod evaluation were performed in order to evaluate the sensorimotor function. First, the negative geotaxis evaluation was carried out on P11, P18, P25, and P32, and specifically, the evaluation of sensorimotor behaviors was analyzed by placing a head of the white rat on an inclined plate to face downward according to the publicly-known method in the related art, and reporting the time taken for the head to face the rear side of the inclined surface.

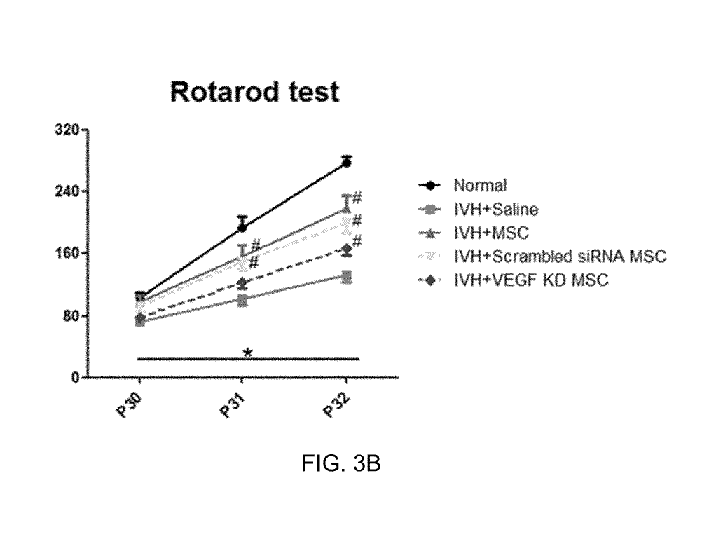

As a result of the evaluations, as illustrated in FIG. 3A, in the case of the control in which intraventricular hemorrhage was caused (IVH+Saline), severe motor function damage was observed as compared to the normal control. However, in the case of the general mesenchymal stem cell transplantation group (IVH+MSC) or the transplantation group of mesenchymal stem cells transfected with the scrambled siRNA (IVH-scrambled siRNA MSC), it was confirmed that the damaged motor ability was remarkably improved. In contrast, in the group into which mesenchymal stem cells, in which the expression of the VEGF was inhibited, were transplanted (IVH+VEGF KD MSC), it was confirmed that the effects of improving the motor ability were not exhibited.

Next, the rotarod evaluation was carried out on each of P30, P31, and P32. As a result, when the rotarod evaluation was initially carried out on P30, a remarkable difference between the groups was not exhibited, but in the case of the normal control (Normal), on P31 and P32, the time taken to fall from a bar was remarkably increased due to the learning effect, whereas in the case of the control in which intraventricular hemorrhage was caused (IVH+Saline), on P31 and P32, the time taken to fall from the bar was measured to be remarkably short as compared to the normal control. In contrast, in the case of the general mesenchymal stem cell transplantation group (IVH+MSC) or the transplantation group of mesenchymal stem cells transfected with the scrambled siRNA (IVH−scrambled siRNA MSC), it was confirmed that the damaged motor function was remarkably improved, but in the group into which mesenchymal stem cells, in which the expression of the VEGF was inhibited, were transplanted (IVH+VEGF KD MSC), it was confirmed that the improved effects as described above were not exhibited.

Through the results, it could be seen that the expression of the VEGF in the mesenchymal stem cells had an important influence on the effects of treating intraventricular hemorrhage caused by mesenchymal stem cells.

### Example 4

### Analysis of Reactive Gliosis, Apoptosis, and Myelination According to Inhibition of Expression of VEGF

4-1. Analysis of Reactive Gliosis

In order to analyze reactive gliosis in tissues around the cerebral ventricle according to the inhibition of the expression of the VEGF in mesenchymal stem cells, cells stained with glial fibrillary acidic protein (GFAP) were observed through an immunohistochemical staining method and compared at a quantitative level.

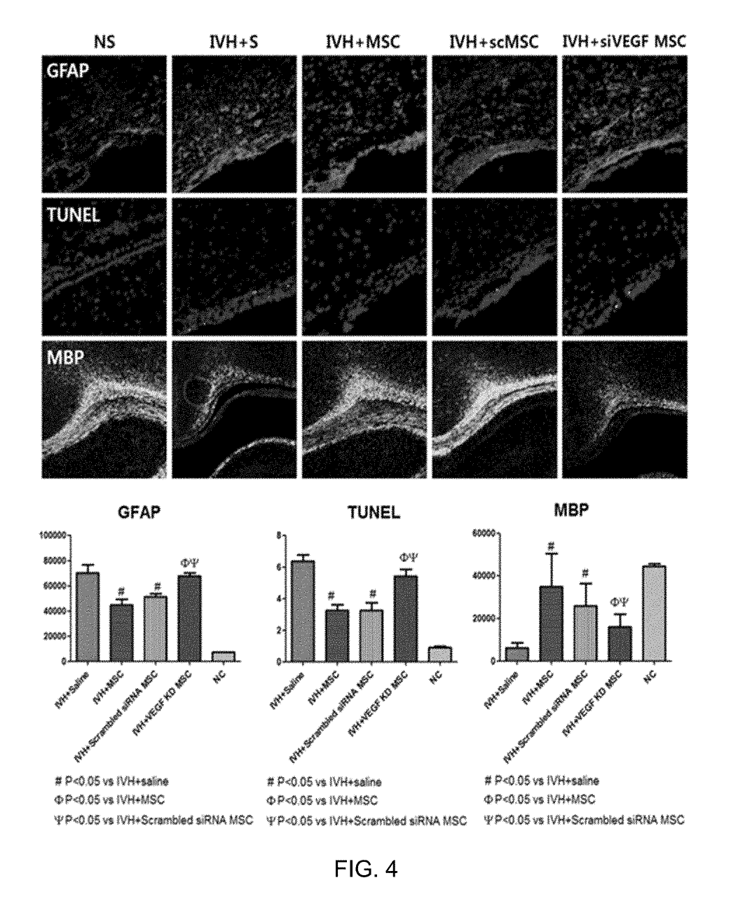

As a result, as illustrated in FIG. 4, it was confirmed that in the case of the control in which intraventricular hemorrhage was caused (IVH+S), the degree of staining of GFAP shown to be red was increased, but in the case of the general mesenchymal stem cell transplantation group (IVH+MSC) and the group into which mesenchymal stem cells transfected with the scrambled siRNA were transplanted (IVH+scMSC), the degree of staining of GFAP was decreased. In contrast, it was confirmed that in the case of the group into which mesenchymal stem cells, in which the expression of the VEGF was inhibited, were transplanted (IVH+siVEGF MSC), the effects of decreasing as described above were not exhibited.

4-2. Analysis of Apoptosis

In order to confirm the degree of apoptosis according to the inhibition of the expression of the VEGF in mesenchymal stem cells after intraventricular hemorrhage, terminal deoxynucleotidyl transferase dUTP nick end labeling (TUNEL) positive cells stained with a TUNEL reagent were observed by performing a TUNEL analysis using tissues around the cerebral ventricle on P32, and the observation was quantitatively analyzed.

As a result, as illustrated in FIG. 4, it was confirmed that in the case of the control in which intraventricular hemorrhage was caused (IVH+S), the number of TUNEL-positive cells was remarkably increased as compared to that of the normal control (NC), whereas in the case of the group into which general mesenchymal stem cells were transplanted (IVH+MSC) and the group into which mesenchymal stem cells transfected with the scrambled siRNA were transplanted (IVH+scMSC), the number of TUNEL positive cells was remarkably decreased. In contrast, it was confirmed that in the case of the group into which mesenchymal stem cells, in which the expression of the VEGF was inhibited, were transplanted (IVH+siVEGF MSC), the apoptosis-suppressive effects as described above were not exhibited.

4-3. Analysis of Myelination

Finally, immunostaining using a Myelin Basic Protein (MBP) antibody was performed in order to evaluate the degree of myelination in tissues around the cerebral ventricle.

As a result, as illustrated in FIG. 4, it was shown that in the case of the control in which intraventricular hemorrhage was caused (IVH+S), the expression of an MBP protein was remarkably decreased as compared to the normal control (NC). However, it was confirmed that in the case of the group into which general mesenchymal stem cells were transplanted (IVH+MSC) and the group into which mesenchymal stem cells transfected with the scrambled siRNA were transplanted (IVH+scMSC), the myelination of nerve cells was improved through an increase in expression level of MBP. In contrast, it was confirmed that in the case of the group into which mesenchymal stem cells, in which the expression of the VEGF was inhibited, were transplanted (IVH+siVEGF MSC), the improved myelination effects as described above were not exhibited.

### Example 5

### Analysis of Inflammations of Tissues around Cerebral Ventricle

In order to verify whether the transplanted mesenchymal stem cells alleviate brain inflammations caused by intraventricular hemorrhage and investigate effects of the VEGF in this case, on P32, the levels of IL-1α, IL-1β, IL-6, and TNF-α, which are inflammatory cytokines from a homogeneous suspension in tissues around the cerebral ventricle, were measured, and the number of ED-1-positive cells in brain coronal sections was measured and analyzed.

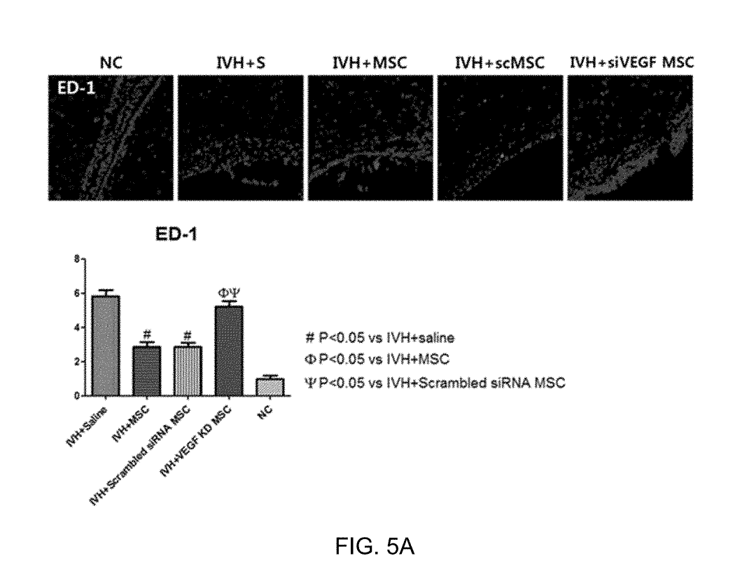

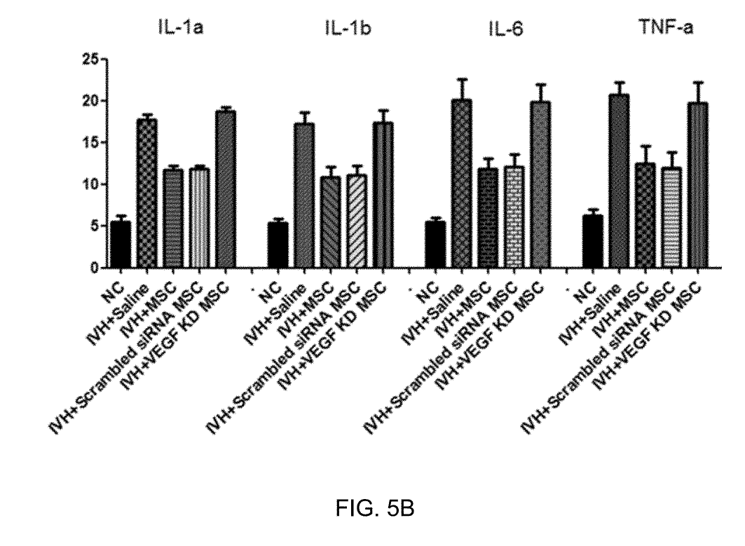

As a result, as illustrated in FIGS. 5A and 5B, it was shown that in the case of the control in which intraventricular hemorrhage was caused (IVH+S), both the number of ED-1-positive cells in tissues around the cerebral ventricle and the levels of inflammatory cytokines were remarkably increased as compared to the normal control (NC). However, it was confirmed that in the case of the group into which general mesenchymal stem cells were transplanted (IVH+MSC) and the group into mesenchymal stem cells transfected with the scrambled siRNA were transplanted (IVH+scMSC), the number of ED-1-positive cells and the levels of inflammatory cytokines were decreased, whereas in the case of the group into which mesenchymal stem cells, in which the expression of the VEGF was inhibited, were transplanted (IVH+siVEGF MSC), this effect of decreasing was not exhibited.

Through the results, it was confirmed that mesenchymal stem cells had nerve cell protective effects in intraventricular hemorrhage, and it could be seen that the expression of the VEGF in the mesenchymal stem cells had an important influence on these effects. Accordingly, the result proves that the evaluation of the expression level of the VEGF has an important meaning in selecting a high efficacy mesenchymal stem cell for treating intraventricular hemorrhage in premature infants.

### Example 6

### Prediction/Selection of High Efficacy Stem Cell by Analysis of Level of VEGF

In order to verify whether the nerve cell protective efficacy of mesenchymal stem cells is shown differently depending on the concentration level of the VEGF, an in vitro model of intraventricular hemorrhage was prepared by treating nerve cells obtained after carrying out a primary neuronal culture from the embryo brains of mice with thrombin. After mesenchymal stem cells exhibiting different expression levels of the VEGF in nerve cells having induced apoptosis were treated lot by lot, the viability of the cells was measured according to the level of the VEGF secreted in each lot.

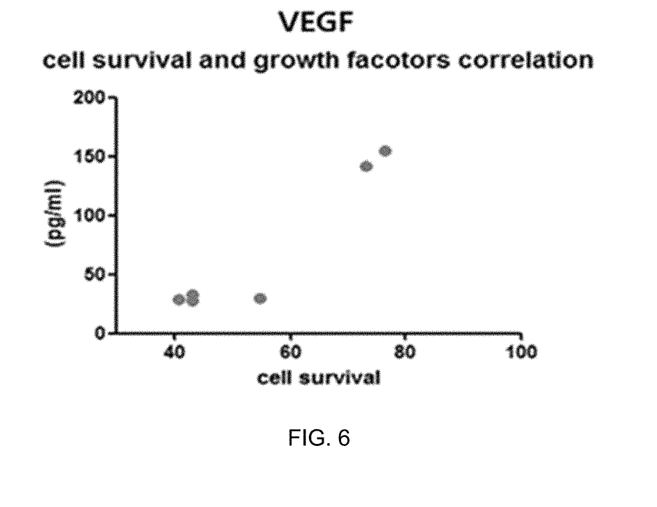

As a result, as illustrated in FIG. 6, it was confirmed that when the VEGF was expressed at a concentration of 50 pg/ml or more, more preferably, when mesenchymal stem cells expressing 100 pg/ml or more were treated, the viability of cells (cell survival) was remarkably increased.

The present inventors confirmed that the VEGF is an important factor which mediates the nerve cell protective effects of mesenchymal stem cells in the treatment of intraventricular hemorrhage since the present inventors confirmed that when mesenchymal stem cells in which the expression of the VEGF was specifically inhibited were transplanted into white rats in which intraventricular hemorrhage was caused, the treatment effects of intraventricular hemorrhage were not exhibited, and increases in reactive gliosis, apoptosis, and inflammation response, and a phenomenon of a decrease in myelination were not alleviated unlike a case where general mesenchymal stem cells were transplanted, and the present inventors elucidated a correlation between the expression level of VEGF and an ability to protect nerve cells in mesenchymal stem cells. Thus, a high efficacy stem cell having an excellent ability to protect nerve cells can be efficiently selected by measuring the level of the VEGF in the selection of mesenchymal stem cells to evaluate the concentration, and it is expected that the method and a high efficacy stem cell selected by the method can be useful in the treatment of various cerebrovascular diseases including intraventricular hemorrhage in a newly born baby.

The above-described description of the present disclosure is provided for illustrative purposes, and the person skilled in the art to which the present disclosure pertains will understand that the present disclosure can be easily modified into other specific forms without changing the technical spirit or essential features of the present disclosure. Therefore, it should be understood that the above-described Examples are only illustrative in all aspects and not restrictive.

What is claimed is:

1. A method for selecting a high efficacy stem cell for treating a cerebrovascular disease, the method comprising:
measuring an expression level of a vascular endothelial growth factor (VEGF).

2. The method according to claim 1, wherein the method comprises:
(a) culturing stem cells;
(b) measuring a concentration of a VEGF in a culture solution of the stem cells in Step (a); and
(c) evaluating an ability to protect nerve cells based on the measured concentration.

3. The method according to claim 2, wherein the evaluation of the ability to protect nerve cells is determined to be high efficacy when the measured concentration of the VEGF is 50 pg/ml or more.

4. The method according to claim 3, wherein the evaluation of the ability to protect nerve cells is determined to be high efficacy when the measured concentration of the VEGF is 100 pg/ml or more.

5. The method according to claim 1, wherein the cerebrovascular disease is intraventricular hemorrhage (IVH) in a newly born baby.

6. The method according to claim 1, wherein the high efficacy is an ability to protect nerve cells.

7. The method according to claim 1, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

8. The method according to claim 7, wherein the mesenchymal stem cell is derived from umbilical cord, umbilical cord blood, bone marrow, fat, muscles, nerves, skin, amnion, or placenta.

9. A high efficacy stem cell for treating a cerebrovascular disease, which is selected by the method of claim 1.

10. The high efficacy stem cell according to claim 9, wherein the cerebrovascular disease is intraventricular hemorrhage (IVH) in a newly born baby.

11. The high efficacy stem cell according to claim 9, wherein the high efficacy is an ability to protect nerve cells.

12. The high efficacy stem cell according to claim 9, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

13. The high efficacy stem cell according to claim 12, wherein the mesenchymal stem cell is derived from umbilical cord, umbilical cord blood, bone marrow, fat, muscles, nerves, skin, amnion, or placenta.

14. A method for treating a cerebrovascular disease comprising:
administering to a subject in need thereof an effective amount of the high efficacy stem cell of claim 9.

15. The method according to claim 14, wherein the cerebrovascular disease is intraventricular hemorrhage (IVH) in a newly born baby.

16. The method according to claim 14, wherein the high efficacy is an ability to protect nerve cells.

17. The method according to claim 14, wherein the stem cell is a stem cell selected from the group consisting of a mesenchymal stem cell, a human tissue-derived mesenchymal stromal cell, a human tissue-derived mesenchymal stem cell, a multipotent stem cell, and an amniotic epithelial cell.

18. The method according to claim 17, wherein the mesenchymal stem cell is derived from umbilical cord, umbilical cord blood, bone marrow, fat, muscles, nerves, skin, amnion, or placenta.
